SEQUENCE LISTING (52 sequences)

SEQ ID NO: 1 - protein, 58 aa, Artificial Sequence
  Col2 binding protein 211190
NAASHDEAQLSALVEIWDLPNLTTRQKQKFIYSLQRDPSVSKEVLGEAQKLNDSQAPK

SEQ ID NO: 2 - protein, 58 aa, Artificial Sequence
  Col2 binding protein 211261
IAAKFDEAQSAADSEILHLPNLTEQQRHYFRRWLSDDPSVSTHILTQAQHLNDDQAPK

SEQ ID NO: 3 - protein, 58 aa, Artificial Sequence
  Col2 binding protein; e.g. 211138 modul 1
IAAKFDEAQYAAFYEIWHLPNLTEEQREAFIASLADDPSVSLEVLGEAQKLNDSQAPK

SEQ ID NO: 4 - protein, 58 aa, Artificial Sequence
  Col2 binding protein, e.g. 209130 modul 1
IAAKFDEAQIAAFWEIYHLPNLTEEQRTAFIWSLYDDPSVSLEVLGEAQKLNDSQAPK

SEQ ID NO: 5 - protein, 58 aa, Artificial Sequence
  Col2 binding protein
  Xaa (42)..(42): may be replaced by any amino acid, preferably Leu, Ala, or Thr
  Xaa (49)..(49): may be replaced by any amino acid, preferably Ile, Arg, or Glu
  Xaa (53)..(53): may be replaced by any amino acid, preferably Leu, Lys, or Tyr
IAAKFDEAQQLAFYQILHLENLTEEQRNAFIQSLRDDPSVSXEVLGEAXKLNXSQAPK

SEQ ID NO: 6 - protein, 58 aa, Artificial Sequence
  Col2 binding protein; e.g. 210987 modul 2
IAAKFDEAQQLAFYQILHLENLTEEQRNAFIQSLRDDPSVSLEVLGEAIKLNLSQAPK

SEQ ID NO: 7 - protein, 58 aa, Artificial Sequence
  Col2 binding protein; e.g. 211027 modul 2
IAAKFDEAQQLAFYQILHLENLTEEQRNAFIQSLRDDPSVSAEVLGEARKLNKSQAPK

SEQ ID NO: 8 - protein, 58 aa, Artificial Sequence
  Col2 binding protein; e.g. 210992 modul 2
IAAKFDEAQQLAFYQILHLENLTEEQRNAFIQSLRDDPSVSTEVLGEAEKLNYSQAPK

SEQ ID NO: 9 - protein, 58 aa, Artificial Sequence
  Col2 binding protein
  Xaa (42)..(42): may be any amino acid, preferably Leu or Pro
  Xaa (43)..(43): may be any amino acid, preferably Glu or Thr
  Xaa (47)..(47): may be any amino acid, preferably His or Thr
  Xaa (50)..(50): may be any amino acid, preferably Gln, Arg, or Leu
IAAKFDEAQQAAFYEILHLPNLTEQQRNYFIQWLRDDPSVSXXVLGXAQXLNDSQAPK

SEQ ID NO: 10 - protein, 58 aa, Artificial Sequence
  Col2 binding protein; e.g. modul 1 of 210987, 210992, 211027
IAAKFDEAQQAAFYEILHLPNLTEQQRNYFIQWLRDDPSVSLEVLGTAQRLNDSQAPK

SEQ ID NO: 11 - protein, 58 aa, Artificial Sequence
  Col2 binding protein, e.g. modul of 211519, or modul 2 of 209130 (210484), 211138
IAAKFDEAQQAAFYEILHLPNLTEQQRNYFIQWLRDDPSVSPTVLGTAQQLNDSQAPK

SEQ ID NO: 12 - protein, 58 aa, Artificial Sequence
  Col2 binding protein, e.g. modul 2 of 210585
IAAKFDEAQQAAFYEILHLPNLTEQQRNYFIQWLRDDPSVSLEVLGHAQLLNDSQAPK

SEQ ID NO: 13 - protein, 58 aa, Artificial Sequence; identical to SEQ ID NO: 9
  Col2 binding protein
  Xaa (42)..(42): may be any amino acid, preferably Leu or Pro
  Xaa (43)..(43): may be any amino acid, preferably Glu or Thr
  Xaa (47)..(47): may be any amino acid, preferably His or Thr
  Xaa (50)..(50): may be any amino acid, preferably Leu, Gln, or Arg

SEQ ID NO: 14 - protein, 58 aa, Artificial Sequence
  Col2 binding protein, e.g.modul 2 of 210970, 210972, 210973, modul 1 of 210971
IAAKFDEAQQAADKEILHLPNLTEQQRNYFRQWLRDDPSVSLEVLGHAQLLNDSQAPK

SEQ ID NO: 15 - protein, 58 aa, Artificial Sequence
  Col2 binding protein, e.g. 211519delFc
IAAKFDEAQQAADKEILHLPNLTEQQRNYFRQWLRDDPSVSPTVLGTAQQLNDSQAPK

SEQ ID NO: 16 - protein, 58 aa, Artificial Sequence
  Col2 binding protein
  Xaa (13)..(13): may be any amino acid, preferably Asp or Phe
  Xaa (29)..(29)<223>  may be any amino acid, preferably Lysor Ala
  Xaa (31)..(31): may be any amino acid, preferably Arg or Ile
IAAKFDEAQWAAXLEIVHLPNLTEEQRLXFXYSLRDDPSVSLEVLGEAQKLNDSQAPK

SEQ ID NO: 17 - protein, 58 aa, Artificial Sequence
  Col2 binding protein, e.g.modul 1 of 210585
IAAKFDEAQWAAFLEIVHLPNLTEEQRLAFIYSLRDDPSVSLEVLGEAQKLNDSQAPK

SEQ ID NO: 18 - protein, 58 aa, Artificial Sequence
  Col2 binding protein, e.g.modul 1 of 210972
IAAKFDEAQWAADLEIVHLPNLTEEQRLKFIYSLRDDPSVSLEVLGEAQKLNDSQAPK

SEQ ID NO: 19 - protein, 58 aa, Artificial Sequence
  Col2 binding protein, e.g.modul 2 of 210971
IAAKFDEAQWAAFLEIVHLPNLTEEQRLKFRYSLRDDPSVSLEVLGEAQKLNDSQAPK

SEQ ID NO: 20 - protein, 58 aa, Artificial Sequence
  Col2 binding protein, e.g.modul 1 of 210970
IAAKFDEAQWAAFLEIVHLPNLTEEQRLKFIYSLRDDPSVSLEVLGEAQKLNDSQAPK

SEQ ID NO: 21 - protein, 58 aa, Artificial Sequence
  Col2 binding protein, e.g.modul 1 of 210973
IAAKFDEAQWAADLEIVHLPNLTEEQRLKFRYSLRDDPSVSLEVLGEAQKLNDSQAPK

SEQ ID NO: 22 - protein, 56 aa, Artificial Sequence
  Col2 binding protein, e.g. modul of 210484 (209130)
AKFDEAQIAAFWEIYHLPNLTEEQRTAFIWSLYDDPSVSLEVLGEAQKLNDSQAPK

SEQ ID NO: 23 - protein, 56 aa, Artificial Sequence
  Col2 binding protein, e.g.modul of 211519delFc
AKFDEAQQAADKEILHLPNLTEQQRNYFRQWLRDDPSVSPTVLGTAQQLNDSQAPK

SEQ ID NO: 24 - protein, 56 aa, Artificial Sequence
  Col2 binding protein, e.g.  211261
AKFDEAQSAADSEILHLPNLTEQQRHYFRRWLSDDPSVSTHILTQAQHLNDDQAPK

SEQ ID NO: 25 - protein, 56 aa, Artificial Sequence
  Col2 binding protein, e.g.modul 1 of 210987, 210992, 211027
AKFDEAQQAAFYEILHLPNLTEQQRNYFIQWLRDDPSVSLEVLGTAQRLNDSQAPK

SEQ ID NO: 26 - protein, 56 aa, Artificial Sequence
  Col2 binding protein, e.g. 211190
ASHDEAQLSALVEIWDLPNLTTRQKQKFIYSLQRDPSVSKEVLGEAQKLNDSQAPK

SEQ ID NO: 27 - protein, 116 aa, Artificial Sequence
  Col2 binding protein (dimer) 209130 (210484)
IAAKFDEAQIAAFWEIYHLPNLTEEQRTAFIWSLYDDPSVSLEVLGEAQKLNDSQAPKIAAKFDEAQQAAFYEILHLPNLTEQQRNYFIQWLRDDPSVSPTVLGTAQQLNDSQAPK

SEQ ID NO: 28 - protein, 116 aa, Artificial Sequence
  Col2 binding protein (dimer) 210970
IAAKFDEAQWAAFLEIVHLPNLTEEQRLKFIYSLRDDPSVSLEVLGEAQKLNDSQAPKIAAKFDEAQQAADKEILHLPNLTEQQRNYFRQWLRDDPSVSLEVLGHAQLLNDSQAPK

SEQ ID NO: 29 - protein, 116 aa, Artificial Sequence
  Col2 binding protein (dimer) 210971
IAAKFDEAQWAAFLEIVHLPNLTEEQRLKFRYSLRDDPSVSLEVLGEAQKLNDSQAPKIAAKFDEAQQAADKEILHLPNLTEQQRNYFRQWLRDDPSVSLEVLGHAQLLNDSQAPK

SEQ ID NO: 30 - protein, 116 aa, Artificial Sequence
  Col2 binding protein (dimer) 210972
IAAKFDEAQWAADLEIVHLPNLTEEQRLKFIYSLRDDPSVSLEVLGEAQKLNDSQAPKIAAKFDEAQQAADKEILHLPNLTEQQRNYFRQWLRDDPSVSLEVLGHAQLLNDSQAPK

SEQ ID NO: 31 - protein, 116 aa, Artificial Sequence
  Col2 binding protein (dimer) 210973
IAAKFDEAQWAADLEIVHLPNLTEEQRLKFRYSLRDDPSVSLEVLGEAQKLNDSQAPKIAAKFDEAQQAADKEILHLPNLTEQQRNYFRQWLRDDPSVSLEVLGHAQLLNDSQAPK

SEQ ID NO: 32 - protein, 116 aa, Artificial Sequence
  Col2 binding protein (dimer) 210992
IAAKFDEAQQAAFYEILHLPNLTEQQRNYFIQWLRDDPSVSLEVLGTAQRLNDSQAPKIAAKFDEAQQLAFYQILHLENLTEEQRNAFIQSLRDDPSVSTEVLGEAEKLNYSQAPK

SEQ ID NO: 33 - protein, 116 aa, Artificial Sequence
  Col2 binding protein (dimer) 211027
IAAKFDEAQQAAFYEILHLPNLTEQQRNYFIQWLRDDPSVSLEVLGTAQRLNDSQAPKIAAKFDEAQQLAFYQILHLENLTEEQRNAFIQSLRDDPSVSAEVLGEARKLNKSQAPK

SEQ ID NO: 34 - protein, 116 aa, Artificial Sequence
  Col2 binding protein (dimer) 211138
IAAKFDEAQYAAFYEIWHLPNLTEEQREAFIASLADDPSVSLEVLGEAQKLNDSQAPKIAAKFDEAQQAAFYEILHLPNLTEQQRNYFIQWLRDDPSVSPTVLGTAQQLNDSQAPK

SEQ ID NO: 35 - protein, 116 aa, Artificial Sequence
  Col2 binding protein (dimer) 210585
IAAKFDEAQWAAFLEIVHLPNLTEEQRLAFIYSLRDDPSVSLEVLGEAQKLNDSQAPKIAAKFDEAQQAAFYEILHLPNLTEQQRNYFIQWLRDDPSVSLEVLGHAQLLNDSQAPK

SEQ ID NO: 36 - protein, 116 aa, Artificial Sequence
  Col2 binding protein (dimer) 210987
IAAKFDEAQQAAFYEILHLPNLTEQQRNYFIQWLRDDPSVSLEVLGTAQRLNDSQAPKIAAKFDEAQQLAFYQILHLENLTEEQRNAFIQSLRDDPSVSLEVLGEAIKLNLSQAPK

SEQ ID NO: 37 - protein, 116 aa, Artificial Sequence
  Col2 binding protein (dimer) 211519
IAAKFDEAQQAAFYEILHLPNLTEQQRNYFIQWLRDDPSVSPTVLGTAQQLNDSQAPKIAAKFDEAQQAAFYEILHLPNLTEQQRNYFIQWLRDDPSVSPTVLGTAQQLNDSQAPK

SEQ ID NO: 38 - protein, 126 aa, Artificial Sequence
  Col2 binding protein (dimer of  211261) (CID211999)
IAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKSAWSHPQFEK

SEQ ID NO: 39 - protein, 146 aa, Artificial Sequence
  Col2 binding protein (dimer of  211261) (CID212000)
IAAKFDEAQSAADSEILHLPNLTEQQRHYFRRWLSDDPSVSTHILTQAQHLNDDQAPKGGGGSGGGGSGGGGSGGGGSIAAKFDEAQSAADSEILHLPNLTEQQRHYFRRWLSDDPSVSTHILTQAQHLNDDQAPKSAWSHPQFEK

SEQ ID NO: 40 - protein, 126 aa, Artificial Sequence
  Col2 binding protein (dimer of  SEQ ID NO: 15) (CID212705)
IAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKSAWSHPQFEK

SEQ ID NO: 41 - protein, 242 aa, Artificial Sequence
  Col2 binding protein (tetramer of  211261) (CID212001)
IAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKIAAKFDEAQSAADSEILHLPNLTEQQRHYFRRWLSDDPSVSTHILTQAQHLNDDQAPKSAWSHPQFEK

SEQ ID NO: 42 - protein, 246 aa, Artificial Sequence
  Col2 binding protein (tetramer of  SEQ ID NO: 15) (CID212704)
IAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKGGGSIAAKFDEAQQAADKEILHLPNLTEQQRNYFRQWLRDDPSVSPTVLGTAQQLNDSQAPKIAAKFDEAQQAADKEILHLPNLTEQQRNYFRQWLRDDPSVSPTVLGTAQQLNDSQAPKSAWSHPQFEK

SEQ ID NO: 43 - protein, 58 aa, Artificial Sequence
  Col2 binding protein
  Xaa (4)..(4): may be any amino acid, preferably Ser
  Xaa (5)..(5): may be any amino acid, preferably His
  Xaa (7)..(7): may be any amino acid, preferably Glu
  Xaa (10)..(10): may be any amino acid, preferably Leu
  Xaa (13)..(13): may be any amino acid, preferably Leu
  Xaa (14)..(14): may be any amino acid, preferably Val
  Xaa (17)..(17): may be any amino acid, preferably Trp
  Xaa (18)..(18): may be any amino acid, preferably Asp
NAAXXDXAQXSAXXEIXXLPNLTTRQKQKFIYSLQRDPSVSKEVLGEAQKLNDSQAPK

SEQ ID NO: 44 - protein, 58 aa, Artificial Sequence; identical to SEQ ID NO: 43
  Col2 binding protein
  misc_feature (4)..(5): Xaa can be any naturally occurring amino acid
  misc_feature (7)..(7): Xaa can be any naturally occurring amino acid
  misc_feature (10)..(10): Xaa can be any naturally occurring amino acid
  misc_feature (13)..(14): Xaa can be any naturally occurring amino acid
  misc_feature (17)..(18): Xaa can be any naturally occurring amino acid
  Xaa (42)..(42): may be any amino acid, preferably Thr
  Xaa (43)..(43): may be any amino acid, preferably Hs
  Xaa (44)..(44): may be any amino acid, preferably Ile
  Xaa (46)..(46): may be any amino acid, preferably Thr
  Xaa (47)..(47): may be any amino acid, preferably Gln
  Xaa (50)..(50): may be any amino acid, preferably His
  Xaa (54)..(54): may be any amino acid, preferably Asp

SEQ ID NO: 45 - protein, 4 aa, Artificial Sequence
  linker
GGGS

SEQ ID NO: 46 - protein, 5 aa, Artificial Sequence
  linker
GGGGS

SEQ ID NO: 47 - protein, 16 aa, Artificial Sequence
  linker
GGGGGSGGGGSGGGGS

SEQ ID NO: 48 - protein, 20 aa, Artificial Sequence
  linker
GGGGSGGGGSGGGGSGGGGS

SEQ ID NO: 49 - protein, 58 aa, Artificial Sequence
  non Ig binding protein
IAAQHDKIQQAADKEILHLPNLTEEQRNKFRQSLRDDPSVSAEILAEAKKLNDAQAPK

SEQ ID NO: 50 - protein, 56 aa, Artificial Sequence
  non Ig binding protein
AQHDKIQQAADKEILHLPNLTEEQRNKFRQSLRDDPSVSAEILAEAKKLNDAQAPK

SEQ ID NO: 51 - protein, 1486 aa, Artificial Sequence
  Type II collagen (Col2)
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 52 - protein, 58 aa, Artificial Sequence
  Type 2 Collagen binding protein
  Xaa (10)..(10): Xaa is selected from Ser and Gln
  Xaa (14)..(14): Xaa is selected from Ser and Gln
  Xaa (28)..(28): Xaa is selected from His and Asn
  Xaa (32)..(32): Xaa is selected from Arg and Gln
  Xaa (35)..(35): Xaa is selected from Ser and Arg
  Xaa (42)..(42): Xaa is selected from Thr and Pro
  Xaa (43)..(43): Xaa is selected from His and Thr
  Xaa (44)..(44): Xaa is selected from Ile and Val
  Xaa (46)..(46): Xaa is selected from Thr and Gly
  Xaa (47)..(47): Xaa is selected from Thr and Gln
  Xaa (50)..(50): Xaa is selected from His and Gln
  Xaa (54)..(54): Xaa is selected from Ser and Asp
IAAKFDEAQXAADXEILHLPNLTEQQRXYFRXWLXDDPSVSXXXLXXAQXLNDXQAPK

(19) United States
(12) Patent Application Publication
(10) Pub. No.: US 2023/0416345 A1
(43) Pub. Date: Dec. 28, 2023

(54) NEW TYPE II COLLAGEN BINDING PROTEINS

(71) Applicant: Navigo Proteins GmbH, Halle/Saale (DE)

(72) Inventors: Eva Bosse-Doenecke, Halle/Saale (DE); Manja Gloser-Braeunig, Halle/Saale (DE); Jonathan Lotze, Halle/Saale (DE); Hanna Bobolowski, Halle/Saale (DE); Erik Fiedler, Halle/Saale (DE)

(73) Assignee: Navigo Proteins GmbH, Halle/Saale (DE)

(21) Appl. No.: 18/036,142

(22) PCT Filed: Nov. 9, 2021
(86) PCT No.: PCT/EP2021/081074

§ 371 Date: May 9, 2023

(30) Foreign Application Priority Data
Nov. 9, 2020 (EP) 20206378.0
Apr. 23, 2021 (EP) 21170199.0

(51) Int. Cl. C07K 16/18

(52) U.S. Cl. CPC C07K 16/18; C07K 2317/92; C07K 2317/14

(57) ABSTRACT

The present invention relates to new type II collagen alpha I chain specific binding proteins. The invention further refers to type II collagen binding proteins further fused to or conjugated to a therapeutically or diagnostically active component. Further aspects of the invention cover the use of these type II collagen binding proteins in medicine.

### FIELD OF THE INVENTION

The present invention relates to new type II collagen alpha I chain specific binding proteins. The invention further refers to type II collagen binding proteins further fused to or conjugated to a therapeutically or diagnostically active component. Further aspects of the invention cover the use of these type II collagen binding proteins in medicine.

### BACKGROUND OF THE INVENTION

Collagens are structural proteins forming three-dimensional meshworks. Type II collagen forms homotrimers resulting in fibrillary structures. Among the many different types of collagen, type II collagen forms the basis for articular cartilage and hyaline cartilage. It makes up 85-90% of collagen of articular cartilage and 65% of collagen of the vitreous humor of the eye. Type II collagen is an important clinical target in particular for eye diseases and joint diseases.

Short collagen binding domains derived from bacterial proteins or other naturally occurring collagen interacting proteins were suggested for certain medical applications including collagenopathy, tissue regeneration, heart disease, or cancer.

However, the disadvantage of collagen binding domains derived from bacterial proteins or naturally occurring interacting proteins is a broad cross reactivity for different types of collagen. Diseases related to structures of the eye and of the joints suffer from poor accessibility for medications. Due to low perfusion, parenterally or orally administered diagnostics and therapeutics do not reach the cartilage of the joints or the vitreous of the eye in sufficient amounts. Although a local application to these structures is possible, a disadvantage of the local application is generally that in particular therapeutics are eliminated too quickly from the body over time, maybe even before the substances have a significant pharmaceutical effect. Therapeutic or diagnostic interventions through injections into the vitreous of the eye or into the joint are inconvenient for patients and physicians alike. Such interventions are risky and painful for the patients.

Needless to say that there is a strong need to relief patients and clinics from the imperative frequent and burdening treatments particularly in eye diseases and joint diseases.

Accordingly, there is a need to find a procedure for a more effective treatment and diagnosis, in particular, a more effective drug delivery to the eye or to the joints. One objective of the present invention is the provision of molecules for anchoring a therapeutic agent near the diseased tissue to prolong its beneficial impact.

To overcome the disadvantages of currently known strategies for treating eye diseases and joint diseases, and other diseases, novel collagen specific binding proteins should include characteristics such as specific affinity to type II collagen. In particular in relation to eye diseases and joint diseases, longer residence times would be desirable for a more effective treatment especially in the light of the burdensome and risky application. Longer local retention of the applied substances is therefore highly desirable to mitigate the burden of the procedure by a less frequent intervention.

Hulme et al. (Journal Of Orthopaedic Research 2017, Vol. 36(4), pp. 1238-1247) describe type II collagen-binding Avimer™ domains. Rothenflu et al. (Nature Materials 2008, Vol. 7(3), pp. 248-254) describe a type II collagen ligand having the six amino acid sequence “WYRGRL”. Formica et al. (Journal of Controlled Release 2019, Vol. 295, pp. 118-129) relate to the “WYRGRL” ligand for cartilage-targeting dexamethasone prodrugs. Brown et al. (Acta Biomater. 2019, Vol. 93, pp. 239-257) is a review article on intra-articular targeting of nanomaterials for the treatment of osteoarthritis.

The invention provides novel type II collagen binding molecules for new and improved strategies in the treatment and diagnosis of various diseases. Binding proteins with specificity for type II collagen enable fewer medical interventions and safer therapies in several severe diseases and improve quality of life for patients.

The above-described objectives and advantages are achieved by the subject-matters of the enclosed claims. The present invention meets the needs presented above by providing examples for type II collagen binding proteins. The above overview does not necessarily describe all problems solved by the present invention.

### SUMMARY OF THE INVENTION

The present disclosure provides the following [1] to [12], without being specifically limited thereto:
[1] A type II collagen binding protein comprising an amino acid sequence with at least 80% sequence identity to any one selected from the group of SEQ ID NOs: 1-44, including a type II collagen binding protein comprising an amino acid sequence with at least 80% sequence identity to any one of SEQ ID NOs: 2, 10, 11, 12, 14, and 15, wherein the type II collagen binding protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15.
[2] The binding protein for type II collagen according to item [1], wherein 2, 3, 4, 5, or 6 binding proteins for type II collagen are linked to each other.
[3] The binding protein for type II collagen according to item [2], wherein the binding protein is a homo-multimer or a hetero-multimer.
[4] The binding protein for type II collagen according to item [3] wherein the binding protein comprises an amino acid sequence with at least 80% sequence identity to any one of SEQ ID NOs: 27-42.
[5] The type II collagen binding protein according to any one of items [1]-[4] wherein the type II collagen binding protein has a binding affinity (K_{D}) of less than 10 μM for type II collagen.
[6] A dimeric or multimeric protein comprising a type II collagen binding protein according to any one of items [1]-[5].
[7] The type II collagen binding protein according to any of items [1]-[5], or the dimeric or multimeric protein according to item [6], further fused to or conjugated to at least one therapeutically active moiety, optionally selected from a monoclonal antibody or a fragment thereof, a binding protein, a receptor or receptor domain, a receptor binding ligand or antagonist, an extracellular domain of a receptor or fragments thereof, a non-immunoglobulin binding protein, a cytokine, a chemokine, a cytotoxic compound, an enzyme, or derivatives thereof, or a radionuclide, or any combination of the above.
[8] The type II collagen binding protein according to any of items [1]-[5] and [7], or the dimeric or multimeric protein according to item [6] or [7], further fused to or conjugated to at least one diagnostic moiety, optionally selected from a radionuclide, a fluorescent compound, a photosensitizer, a tag, an enzyme, or a non-immunoglobulin binding protein, or any combination of the above.
[9] The type II collagen binding protein according to any of items [1]-[5], [7] and [8], for use in medicine, preferably for use in the diagnosis or treatment of diseases such as eye diseases or diseases of the joint.
[10] A composition comprising the type II collagen binding protein according to any of items [1]-[5], [7] and [8], or the dimeric or multimeric protein according to any of items [6] to [8].
[11] A pharmaceutical composition for the treatment of diseases comprising the type II collagen binding protein, or the dimeric or multimeric protein, as defined in any of the preceding aspects and a therapeutically or diagnostically acceptable carrier and/or diluent.
[12] A method for the production of a type II collagen binding protein, or the dimeric or multimeric protein, as defined in any of the preceding aspects comprising culturing of a host cell under suitable conditions in order to obtain said type II collagen binding protein and optionally isolating said type II collagen binding protein.

This summary does not necessarily describe all features of the present invention. Other embodiments become apparent from a review of the ensuing detailed description.

### BRIEF DESCRIPTION OF THE FIGURES

FIG. 1A. Amino acid sequences of type II collagen binding proteins. The numbers in the top row refer to the corresponding amino acid position in the Ig binding protein. FIG. 1B. Reference sequences SEQ ID NOs: 2, 10, 11, 12, 14 and 15 having the motif 25Q-29Y-33W.

FIG. 1C. Dimeric type II collagen binding proteins having the motif 25Q-29Y-33W “Monomer 1” refers to the N-terminal located moiety; “monomer 2” refers to the C-terminal located moiety. The monomer domains carrying the motif 25Q-29Y-33W are shown in grey scale.

FIG. 2. Collagen binding analysis via ELISA shows highly specific binding to type II collagen. No binding to type I, type III or type V collagen and unrelated fibronectin domain EDB (67B89) could be observed.

FIG. 2A. Binding analysis of the dimer of SEQ ID NO: 2 (CID211999) (dimer of directly joint monomers).

FIG. 2B. Binding analysis of the dimer of SEQ ID NO: 2 (CID212000) (dimer with linker between the monomers).

FIG. 2C. Binding analysis of the tetramer of SEQ ID NO: 2 (CID212001).

FIG. 2D. Binding analysis of the dimer of SEQ ID NO: 15 (CID212705).

FIG. 2E. Binding analysis of the tetramer of SEQ ID NO: 15 (CID212704).

FIG. 3. Binding analysis via SPR of the tetramer of SEQ ID NO: 2 (CID212001) shows binding to type II collagen with high affinity.

FIG. 4. Binding analysis of 211519 (dimer of SEQ ID NO: 11) via SPR. FIG. 4A. Concentration series of 211519 (SEQ ID NO: 37). multi-cycle Biacore measurement confirms binding to Type II collagen with transient binding signal. FIG. 4B. Equilibrium fit of amplitudes—KD of 780 nM for Type II collagen. FIG. 4C. 211519 (homodimer of SEQ ID NO: 11) is specific for Type II collagen; no binding for Type I collagen, Type III collagen, Type V collagen was observed (analysis via SPR).

FIG. 5. Immunohistochemistry staining on pig eye sections. Type II collagen binding protein of SEQ ID NO: 39 (dimer of SEQ ID NO: 2, linker between monomers, CID212000) shows a staining of retina, inner limiting membrane (arrow) and vitreous body of pig eye.

### DETAILED DESCRIPTION OF THE INVENTION

The proteins of the invention are specifically bound to ocular type II collagen by means of a non-Immunoglobulin-binding protein. The present inventors have developed a solution to meet the ongoing need in the art by providing type II collagen specific proteins comprising an amino acid sequence with at least 80% sequence identity to any one selected from the group of SEQ ID NOs: 1-26, 43, 44. The type II collagen specific proteins as defined herein are functionally characterized by high affinity for type II collagen. In particular, the invention provides type II collagen binding proteins comprising an amino acid sequence with at least 80% sequence identity to any one selected from the group of SEQ ID NOs: 1-44. The novel type II collagen specific proteins may broaden therapeutic and diagnostic options. In some embodiments, at least 2 type II collagen binding proteins are linked to each other to generate multimers, as shown for example in SEQ ID NOs: 27-42.

Before the present invention is described in more detail below, it is to be understood that this invention is not limited to the particular methodology, protocols and reagents described herein as these may vary. It is also to be understood that the terminology used herein is for the purpose of describing particular aspects and embodiments only, and is not intended to limit the scope of the present invention, which is reflected by the appended claims. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new target-specific binding molecules for use in technical applications and in therapy and diagnostics.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims, which follow, unless the context requires otherwise, the word “comprise”, and variants such as “comprises” and “comprising”, was understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, GenBank Accession Number sequence submissions etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing that, with its whole content and disclosure, forms part of the disclosure content of the present specification.

### General Definitions of Important Terms Used in the Application

The term “type II collagen alpha I chain” or “type II collagen” as used herein refers to uniprot Accession Number P02458.2, amino acids 182 to 1241 (SEQ ID NO: 51). The term “type II collagen” comprises all polypeptides which show a sequence identity of at least 80%, 85%, 90%, 95%, 96% or 97% or more, or 100% to SEQ ID NO: 51 and have the functionality of type II collagen. In particular, the term comprises type II collagen from other mammalian species with amino acid sequence identity of at least 80%, 85%, 90%, 95%, 96% or 97% or more, or 100% to SEQ ID NO: 51. In accordance with the reference to type II collagen identified under uniprot Accession Number P02458.2, in preferred embodiments the term “type II collagen” as used herein means human type II collagen.

The term “type II collagen binding protein” or “binding protein for type II collagen” or “Col2 BP” refers to a protein with high affinity binding to type II collagen.

The terms “protein” and “polypeptide” refer to any chain of two or more amino acids linked by peptide bonds, and does not refer to a specific length of the product. Thus, “peptides”, “protein”, “amino acid chain”, or any other term used to refer to a chain of two or more amino acids, are included within the definition of “protein”, and the term “protein” may be used instead of, or interchangeably with, any of these terms. The term “protein” is also intended to refer to the products of post-translational modifications of the polypeptide, which are well known in the art.

The term “modification” or “amino acid modification” refers to a substitution, a deletion, or an insertion of a reference amino acid at a particular position in a parent polypeptide sequence by another amino acid. Given the known genetic code, and recombinant and synthetic DNA techniques, the skilled scientist can readily construct DNAs encoding the amino acid variants.

The terms “binding affinity” and “binding activity” may be used herein interchangeably, and they refer to the ability of a polypeptide to bind to another protein, peptide, or fragment or domain thereof. Binding affinity is typically measured and reported by the equilibrium dissociation constant (K_{D}), which is used to evaluate and rank order strengths of bimolecular interactions.

The term “fusion protein” relates to a protein comprising at least a first protein joined genetically to at least a second protein. A fusion protein is created through joining of two or more genes that originally coded for separate proteins. Fusion proteins may further comprise additional domains that are not involved in binding of the target, such as but not limited to, for example, multimerization moieties, polypeptide tags, polypeptide linkers, half-life extending moieties.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide, for example, to the polypeptide of any of SEQ ID NOs: 1-44. Methods for sequence alignment and sequence comparison algorithms are well known in the art. For example, for determining the extent of an amino acid sequence identity of an arbitrary polypeptide relative to the amino acid sequence of, for example, any of SEQ ID NOs: 1-44, the SIM Local similarity program as known in the art is preferably employed. For multiple alignment analysis, Clustal Omega is preferably used, as known to someone skilled in the art.

### DETAILED DESCRIPTION OF THE EMBODIMENTS OF THIS INVENTION

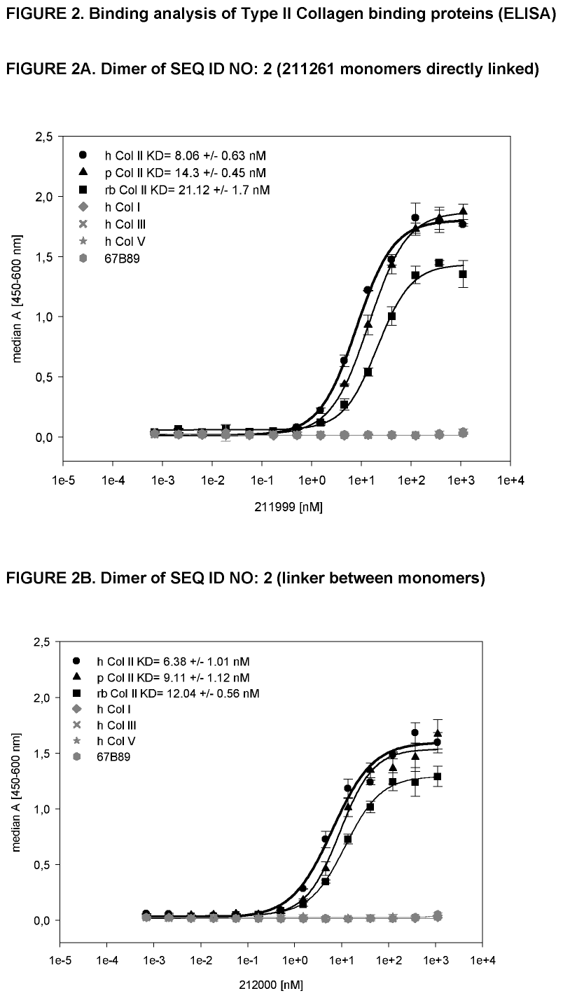

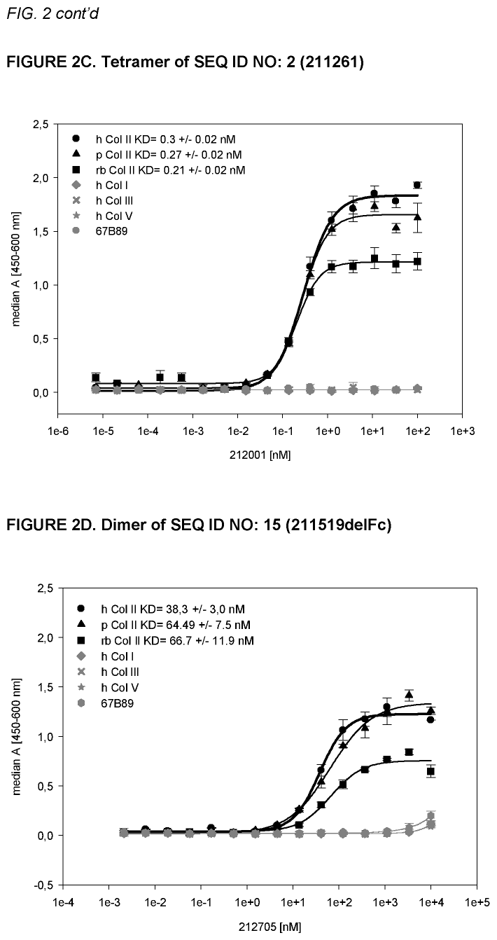

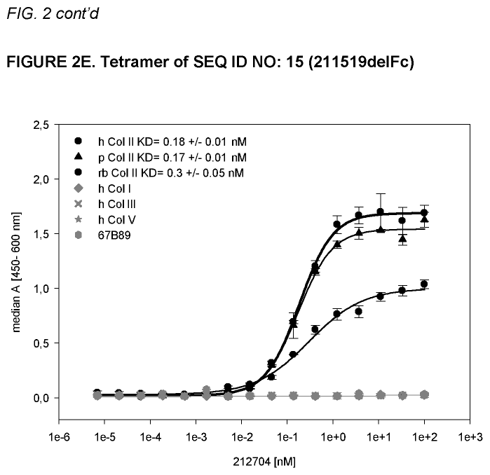

The type II collagen binding protein as defined herein comprises an amino acid sequence with at least 80% sequence identity to any one selected from the group of SEQ ID NOs: 1-44. In particular provided herein is a type II collagen binding protein comprising an amino acid sequence with at least 80% sequence identity to any one of SEQ ID NOs: 2, 10, 11, 12, 14, and 15, wherein the type II collagen binding protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and wherein the type II collagen binding protein has a binding affinity (K_{D}) of less than 10 μM for type II collagen. In various preferred embodiments, the type II collagen binding protein comprises an amino acid sequence with at least 85% or 89.5% sequence identity to any one of SEQ ID NOs: 2, 10, 11, 12, 14, and 15, wherein the type II collagen binding protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and wherein the type II collagen binding protein has a binding affinity (K_{D}) of less than 10 μM for type II collagen. In various other preferred embodiments, the type II collagen binding protein comprises an amino acid sequence with at least 90% sequence identity, preferably any of at least 91%, 92%, 93%, 94%, or 95% sequence identity, more preferably any of at least 96%, 97%, 98%, or 99% sequence identity, to any one of SEQ ID NOs: 2, 10, 11, 12, 14, and 15, wherein the type II collagen binding protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and wherein the type II collagen binding protein has a binding affinity (K_{D}) of less than 10 μM for type II collagen. Such type II collagen binding proteins exhibit specific binding affinity for type II collagen, i.e., no unspecific binding to type I collagen, type III collagen, type V collagen or to a domain of fibronectin (67B89) was detected, as shown in FIG. 2. In various embodiments, a type II collagen binding protein of the present disclosure has no detectable binding affinity to type I collagen, and/or type III collagen, and/or type V collagen and/or to a domain of fibronectin (67B89), as determined by methods for determining binding affinities described elsewhere herein. In preferred embodiments, the method for determining binding affinity to type I collagen, and/or type III collagen, and/or type V collagen and/or a domain of fibronectin (67B89), is any of ELISA, SPR, BLI, KinExA assay, flow cytometry, fluorescence spectroscopy techniques, ITC, analytical ultracentrifugation, RIA or IRMA, and ECL, more preferably any of ELISA, SPR, BLI, KinExA assay, and flow cytometry, even more preferably any of ELISA, SPR, and KinExA assay, still more preferably ELISA or SPR, and most preferred ELISA. In various embodiments, a type II collagen binding protein of the present disclosure has no detectable binding affinity to type I collagen and/or type Ill collagen. In various other embodiments, a type II collagen binding protein of the present disclosure has no detectable binding affinity to type I collagen and type III collagen, and/or type V collagen. In various preferred embodiments, a type II collagen binding protein comprising an amino acid sequence with at least 80% sequence identity to any one of SEQ ID NOs: 2, 10, 11, 12, 14, and 15, wherein the type II collagen protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; has a binding affinity (K_{D}) of less than 1 μM for type II collagen, more preferably less than 500 nM. In other embodiments, a type II collagen binding protein as described above has a binding affinity (K_{D}) of less than 200 nM, or of less than 100 nM, and even of less than 50 nM, for type II collagen.

In various embodiments, a Type II Collagen binding protein has at least 95% amino acid sequence identity to IAAKFDEAQX₁₀AADX₁₄EILHLPNLTEQQRX₂₈YFRX₃₂WLX₃₅DDPSVSX₄₂X₄₃X₄₄LX₄₆X₄₇AQX₅₀LNDX₅₄QAPK (SEQ ID NO: 52) wherein, individually of each other, X₁₀ is selected from S or Q, X₁₄ is selected from S or K, X₂₈ is selected from H or N, X₃₂ is selected from R or Q, X₃₅ is selected from S or R, X₄₂ is selected from T or P, X₄₃ is selected from H or T, X₄₄ is selected from I or V, X₄₆ is selected from T or G, X₄₇ is selected from Q or T, X₅₀ is selected from H or Q, and X₅₄ is selected from S or D. In some embodiments, a Type II Collagen binding protein has at least 95% amino acid sequence identity to SEQ ID NO: 52 wherein, individually of each other, position 25 is Q, position 29 is Y, and position 33 is W.

In various embodiments, a Type II Collagen binding protein has at least 95% amino acid sequence identity to IAAKFDEAQX₁₀AADX₁₄EILHLPNLTEQQRX₂₃YFRX₃₂WLX₃₅DDPSVSX₄₂X₄₃X₄₄LX₄₆X₄₇AQX₅₀LNDX₅₄QAPK (SEQ ID NO: 52) wherein X₁₀ is selected from S or Q, X₁₄ is selected from S or K, X₂₈ is selected from H or N, X₃₂ is selected from R or Q, X₃₅ is selected from S or R, X₄₂ is selected from T or P, X₄₃ is selected from H or T, X₄₄ is selected from I or V, X₄₆ is selected from T or G, X₄₇ is selected from Q or T, X₅₀ is selected from H or Q, and X₅₄ is selected from S or D. In some embodiments, a Type II Collagen binding protein has at least 95% amino acid sequence identity to SEQ ID NO: 52 wherein position 25 is Q, position 29 is Y, and position 33 is W.

In various embodiments, a Type II Collagen binding protein has at least 95% amino acid sequence identity to

(SEQ ID NO: 2, CID 211261)
IAAKFDEAQSAADSEILHLPNLTEQQRHYFRRWLSDDPSVSTHILTQAQH
LNDDQAPK.

The Type II Collagen binding protein of the present invention as disclosed herein has at least 95%, 96%, 97%, 98%, or 99% amino acid sequence identity to the amino acid sequence of SEQ ID NO: 2. In some embodiments, the Type II Collagen binding protein of the present invention as disclosed herein has at least 95%, 96%, 97%, 98%, or 99% amino acid sequence identity to the amino acid sequence of SEQ ID NO: 2 wherein position 25 is Q, position 29 is Y, and position 33 is W.

In various embodiments, the Type II Collagen binding protein has at least 95% amino acid sequence identity to SEQ ID NO: 15, CID 211519delFc. The Type II Collagen binding protein of the present invention as disclosed herein has at least 95%, 96%, 97%, 98%, or 99% amino acid sequence identity to the amino acid sequence of IAAKFDEAQQAADKEILHLPNLTEQQRNYFRQWLRDDPSVSPTVLGTAQQLNDSQAPK to SEQ ID NO: 15. In some embodiments, the Type II Collagen binding protein of the present invention as disclosed herein has at least 95%, 96%, 97%, 98%, or 99% amino acid sequence identity to the amino acid sequence of to SEQ ID NO: 15, provided that position 25 is Q, position 29 is Y, and position 33 is W.

Functional characterization. One embodiment refers to type II collagen binding protein with binding affinity (K_{D}) of less than 10 μM for type II collagen. The type II collagen binding proteins bind type II collagen with measurable binding affinity of less than 1 μM, less than 500 nM, less than 200 nM, less than 100 nM, or less than 50 nM. The appropriate methods are known to those skilled in the art or described in the literature. The methods for determining the binding affinities are known per se and can be selected for instance from the following methods known in the art: enzyme-linked immunosorbent assay (ELISA), surface plasmon resonance (SPR), Bio-layer interferometry (BLI), kinetic exclusion analysis (KinExA assay), flow cytometry, fluorescence spectroscopy techniques, isothermal titration calorimetry (ITC), analytical ultracentrifugation, radioimmunoassay (RIA or IRMA), and enhanced chemiluminescence (ECL). Some of the methods are described in the Examples below. Typically, the dissociation constant K_{D} is determined at 20° C., 25° C., or 30° C. If not specifically indicated otherwise, the K_{D} values recited herein are determined at 25° C. by SPR. The lower the K_{D} value, the greater the binding affinity of the biomolecule for its binding partner. The higher the K_{D} value, the more weakly the binding partners bind to each other.

Structural Characterization of Type II Collagen Binding Proteins.

In various embodiments, a type II collagen binding protein as disclosed herein has at least 80% sequence identity to the amino sequence of any one of SEQ ID NOs: 1-44. A type II collagen binding protein as disclosed herein has at least 81%, 82%, 83%, 84%, 85%, 86%, 87%, 88%, or 89% sequence identity to the amino acid sequence of any one of SEQ ID NOs: 1-44. A type II collagen binding protein as disclosed herein has at least 90%, 91%, 92%, 93%, 94%, 95%, 96%, 97%, 98%, or 99% amino acid sequence identity to the amino acid sequence of any one of SEQ ID NOs: 1-44.

In various embodiments, a type II collagen binding protein as disclosed herein is comprising, or consisting of, a protein that has at least 90% sequence identity to the amino sequence of SEQ ID NO: 1 (CID211190). In various embodiments, a type II collagen binding protein as disclosed herein is comprising, or consisting of, a protein that has at least 90% sequence identity to the amino sequence of SEQ ID NO: 2 (CID211261). In various embodiments, a type II collagen binding protein as disclosed herein is comprising, or consisting of, a protein that has at least 90% sequence identity to the amino sequence of SEQ ID NO: 20 (CID210970). In various embodiments, a type II collagen binding protein as disclosed herein is comprising, or consisting of, a protein that has at least 90% sequence identity to the amino sequence of SEQ ID NO: 15 (CID211519delFc). A type II collagen binding protein as disclosed herein has at least 90%, 91%, 92%, 93%, 94%, 95%, 96%, 97%, 98%, or 99% amino acid sequence identity to the amino acid sequence of any one of SEQ ID NOs: 1-44.

In some embodiments, a type II collagen binding protein is comprising an amino acid sequence with at least 80% identity to the amino acid sequence of SEQ ID NO: 5: IAAKFDEAQQLAFYQILHLENLTEEQRNAFIQSLRDDPSVSX₄₂EVLGEAX₄₉KLNX₅₃SQAPK, wherein X can be any amino acid, preferably X₄₂ is selected from L or A or T, X₄₉ is selected from I, R, or E, X₅₃ is selected from L, K, or Y. Specific examples are provided in SEQ ID NOs: 6, 7, 8, or in the dimers of SEQ ID NOs: 32, 33, 36.

In some embodiments, a type II collagen binding protein is comprising an amino acid sequence with at least 80% identity to the amino acid sequence of SEQ ID NO: 9: IAAKFDEAQQAAFYEILHLPNLTEQQRNYFIQWLRDDPSVSX₄₂X₄₃VLGX₄₇AQX₅₀LNDSQAPK, wherein X can be any amino acid, preferably X₄₂ is selected from L or P, X₄₃ is selected from E or T, X₄₇ is selected from H or T, X₅₀ is selected from Q, L, or R. Specific examples are provided in SEQ ID NOs: 10-12, 25, or in dimers of SEQ ID NOs: 27, 32-37.

In some embodiments, a type II collagen binding protein is comprising an amino acid sequence with at least 80% identity to the amino acid sequence of SEQ ID NO: 13: IAAKFDEAQQAAFYEILHLPNLTEQQRNYFIQWLRDDPSVSX₄₂X₄₃VLGX₄₇AQX₅₀LNDSQAPK, wherein X can be any amino acid, preferably X₄₂ is selected from L or P, X₄₃ is selected from E or T, X₄₇ is selected from H or T, X₅₀ is selected from Q, L, or R. Specific examples are provided in SEQ ID NO: 14, 15, 23, in dimers of SEQ ID NOs: 28, 29, 30, 31, 30, and in the tetramer of SEQ ID NO: 42.

In some embodiments, a type II collagen binding protein is comprising an amino acid sequence with at least 80% identity to the amino acid sequence of SEQ ID NO: 16: IAAKFDEAQWAAX₁₃LEIVHLPNLTEEQRLX₂₉FX₃₁YSLRDDPSVSLEVLGEAQKLNDSQAPK, wherein X can be any amino acid, preferably X₁₃ is selected from D or F, X₂₉ is selected from K or A, X₃₁ is selected from R or I. Specific examples are provided in SEQ ID NOs: 17-21, or in dimers of SEQ ID NO: 28, 29, 30, 31, 35.

In some embodiments, a type II collagen binding protein is comprising an amino acid sequence with at least 80% identity to the amino acid sequence of SEQ ID NO: 43: NAAXXDXAQXSAXXEIXXLPNLTTRQKQKFIYSLQRDPSVSKEVLGEAQKLNDSQAPK.

Specific examples are provided in SEQ ID NOs: 1 and 26.

In some embodiments, a type II collagen binding protein is comprising an amino acid sequence with at least 80% identity to the amino acid sequence of SEQ ID NO: 44: IAAKFDEAQSAADSEILHLPNLTEQQRHYFRRWLSDDPSVSXXXLXXAQXLNDXQAPK.

Specific examples are provided in SEQ ID NOs: 2, 24, in dimers of SEQ ID NO: 38, 39, and in the tetramer of SEQ ID NO: 41.

In various embodiments of the present invention, a type II collagen binding protein as disclosed herein may have a deletion of one or more, preferably two, amino acid residues at the N-terminus of any of SEQ ID NOs: 1-44. In various embodiments of the present invention, a type II collagen binding protein as disclosed herein may have a deletion of one or more, preferably two, amino acid residues at the N-terminus of any of the monomer sequences of SEQ ID NOs: 1-26, 43, and 44, as exemplified by SEQ ID NOs: 22-26. In preferred embodiments of dimeric and multimeric type II collagen binding proteins disclosed herein, only the monomer located at the N-terminus of the dimer or multimer may have a deletion of one or more, preferably two, amino acid residues at the N-terminus of the monomer. In various embodiments of the present invention, a monomer type II collagen binding protein as disclosed herein comprises at least 47 amino acid residues of any of SEQ ID NOs: 1-26, 43 and 44. In various other embodiments of the present invention, a monomer type II collagen binding protein as disclosed herein comprises at least 48 or 49 amino acid residues of any of SEQ ID NOs: 1-26, 43 and 44. In still other embodiments of the present invention, a monomer type II collagen binding protein as disclosed herein comprises at least 50 or 51 amino acid residues of any of SEQ ID NOs: 1-26, 43 and 44. In various preferred embodiments of the present invention, a monomer type II collagen binding protein as disclosed herein comprises at least 52 amino acid residues of any of SEQ ID NOs: 1-26, 43 and 44. In various other preferred embodiments of the present invention, a monomer type II collagen binding protein as disclosed herein comprises at least 53 amino acid residues of any of SEQ ID NOs: 1-26, 43 and 44. More preferably, a monomer type II collagen binding protein as disclosed herein comprises at least 54 or 55 amino acid residues of any of SEQ ID NOs: 1-26, 43 and 44. In particularly preferred embodiments of the present invention, a monomer type II collagen binding protein as disclosed herein comprises at least 56 amino acid residues of any of SEQ ID NOs: 1-26, 43 and 44. In various preferred embodiments of the present invention, a monomer type II collagen binding protein as disclosed herein comprises at least 57 or even 58 amino acid residues of any of SEQ ID NOs: 1-26, 43 and 44.

In various embodiments of the present invention, a type II collagen binding protein as disclosed herein may have a deletion of one or more, preferably two, amino acid residues at the N-terminus of any of the monomer sequences of SEQ ID NOs: 1-26, 43, and 44, as exemplified by SEQ ID NOs: 22-26. In preferred embodiments of dimeric and multimeric type II collagen binding proteins disclosed herein, only the monomer located at the N-terminus of the dimer or multimer may have a deletion of one or more, preferably two, amino acid residues at the N-terminus of the monomer.

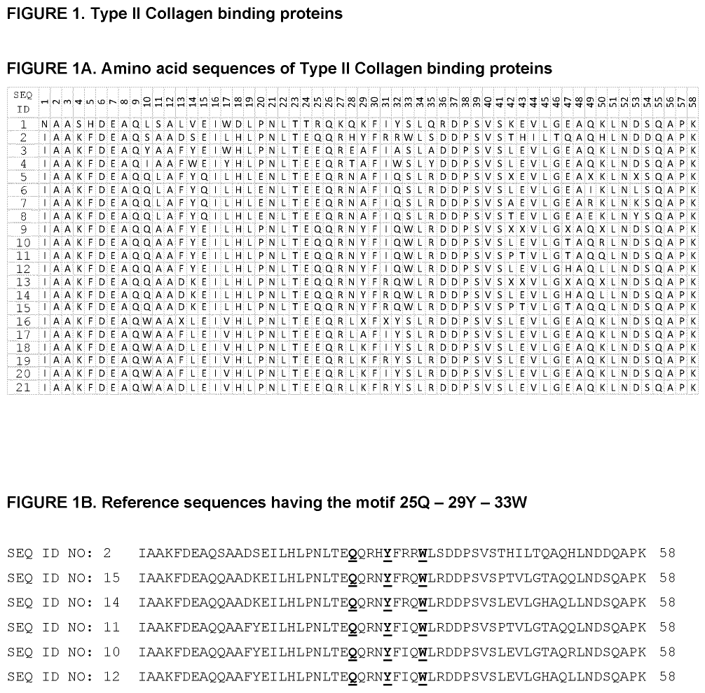

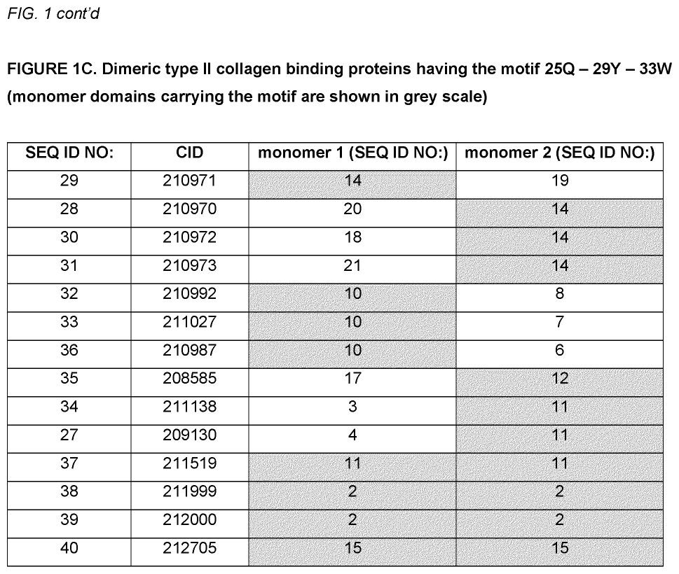

FIG. 1C exemplifies dimeric type II collagen binding proteins of the present invention, wherein at least one of the two monomer domains is characterized by the motif 25Q-29Y-33W. Accordingly, in various embodiments, a dimeric or multimeric type II collagen binding protein of the present invention comprises at least one monomer, which is a type II collagen binding protein comprising an amino acid sequence with at least 80% sequence identity to any one of SEQ ID NOs: 2, 10, 11, 12, 14, and 15, wherein the monomer type II collagen protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15. In various preferred embodiments, the monomer type II collagen binding protein comprised by a dimeric or multimeric type II collagen binding protein of the present invention has a binding affinity (K_{D}) of less than 10 μM for type II collagen, as described elsewhere herein. The monomer type II collagen binding protein comprised by a dimeric or multimeric type II collagen binding protein of the present invention may be located at the N-terminus or at the C-terminus of the dimeric or multimeric type II collagen binding protein.

Multimeric type II collagen binding proteins of the present invention may comprise one or more monomer type II collagen binding proteins with at least 80% sequence identity to any of SEQ ID NOs: 1-26, 43 and 44, and may comprise one or more monomer type II collagen binding proteins with at least 80% sequence identity to any one of SEQ ID NOs: 2, 10, 11, 12, 14, and 15, wherein the monomer type II collagen protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and wherein the monomer type II collagen binding protein has a binding affinity (K_{D}) of less than 10 μM for type II collagen, as described elsewhere herein. The monomer type II collagen binding protein comprised by a multimeric type II collagen binding protein of the present invention may or may not be located at the N-terminus or at the C-terminus of the multimeric type II collagen binding protein.

In various embodiments, a binding protein for type II collagen provided by the present invention comprises an amino acid sequence with at least 80% sequence identity to any one of the dimers of SEQ ID NOs: 27-40, wherein at least one of the two monomers is a type II collagen binding protein having (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and having a binding affinity (K_{D}) of less than 10 μM for type II collagen, as described elsewhere herein. The at least one monomer may be located either at the N-terminus or at the C-terminus of the dimeric type II collagen binding protein with at least 80% sequence identity to any one of SEQ ID NOs: 27-40. In various preferred embodiments, the dimeric type II collagen binding protein comprises an amino acid sequence with at least 85% or 89.5% sequence identity to any one of SEQ ID NOs: 27-40, wherein at least one of the two monomers is a type II collagen binding protein having (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and having a binding affinity (K_{D}) of less than 10 μM for type II collagen, as described elsewhere herein. In still other preferred embodiments, the dimeric type II collagen binding protein comprises an amino acid sequence with at least 90% sequence identity, preferably any of at least 91%, 92%, 93%, 94%, or 95% sequence identity, more preferably any of at least 96%, 97%, 98%, or 99% sequence identity, to any one of SEQ ID NOs: 27-40, wherein at least one of the two monomers is a type II collagen binding protein having (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and having a binding affinity (K_{D}) of less than 10 μM for type II collagen, as described elsewhere herein.

In various embodiments, a binding protein for type II collagen provided by the present invention comprises an amino acid sequence with at least 80% sequence identity to the tetramer of SEQ ID NO: 41 or 42, wherein one, two, three, or all of the four monomers is/are a type II collagen binding protein having (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and having a binding affinity (K_{D}) of less than 10 μM for type II collagen, as described elsewhere herein. One of the up to four monomers may be located either at the N-terminus or at the C-terminus of the tetrameric type II collagen binding protein with at least 80% sequence identity to SEQ ID NO: 41 or 42. In various preferred embodiments, the tetrameric type II collagen binding protein comprises an amino acid sequence with at least 85% or 89.5% sequence identity to SEQ ID NO: 41 or 42, wherein one, two, three or all of the four monomers is/are a type II collagen binding protein having (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and having a binding affinity (K_{D}) of less than 10 μM for type II collagen, as described elsewhere herein. In still other preferred embodiments, the tetrameric type II collagen binding protein comprises an amino acid sequence with at least 90% sequence identity, preferably any of at least 91%, 92%, 93%, 94%, or 95% sequence identity, more preferably any of at least 96%, 97%, 98%, or 99% sequence identity, to SEQ ID NO: 41 or 42, wherein one, two, three or all of the four monomers is/are a type II collagen binding protein having (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and having a binding affinity (K_{D}) of less than 10 μM for type II collagen, as described elsewhere herein. As further disclosed herein, in various embodiments, a dimeric or multimeric type II collagen binding protein of the present invention may comprise one or more linker sequences linking the monomer type II collagen binding proteins comprised by the dimeric or multimeric type II collagen binding protein. In various embodiments, the linker sequence comprises the amino acid sequence GGGS (SEQ ID NO: 45). In various other embodiments, the linker sequence comprises the amino acid sequence GGGGS (SEQ ID NO: 46). In still other embodiments, the linker sequence comprises the amino acid sequence GGGGSGGGGSGGGGS (SEQ ID NO: 47), or comprises the amino acid sequence GGGGSGGGGSGGGGSGGGGS (SEQ ID NO: 48).

The present invention further provides binding proteins for type II collagen with no detectable binding affinity for the Fc domain of immunoglobulin (or lacking binding affinity for the Fc domain of immunoglobulin), wherein the type II collagen binding protein comprises an amino acid sequence with at least 80% sequence identity to any one of SEQ ID NOs: 1-44. In various embodiments, the present invention provides binding proteins for type II collagen with no detectable binding affinity for the Fc domain of immunoglobulins, wherein the type II collagen binding protein comprises an amino acid sequence with at least 80% sequence identity to any one of SEQ ID NOs: 2, 10, 11, 12, 14, and 15, wherein the type II collagen binding protein has (i) a glutamine (Q) at the position corresponding to position 25 in any of SEQ ID NOs: 2, 10, 11, 12, 14 and 15; (ii) a tyrosine (Y) at the position corresponding to position 29 in any of SEQ ID NOs: 2, 10, 11, 12, 14 and 15; and (iii) a tryptophan (W) at the position corresponding to position 33 in any of SEQ ID NOs: 2, 10, 11, 12, 14 and 15; and has a binding affinity (K_{D}) of less than 10 μM for type II collagen, as described elsewhere herein. Type II collagen binding proteins of the present invention with no detectable binding affinity for the Fc domain of immunoglobulins comprise one or more, preferably two or three, of the following amino acid residues in any of SEQ ID NOs: 1-21: aspartate (D) at the position corresponding to position 13 (13D) of any of SEQ ID NOs: 1-21; lysine (K) at the position corresponding to position 14 (14K) of any of SEQ ID NOs: 1-21; and/or arginine (R) at the position corresponding to position 31 (31R) of any of SEQ ID NOs: 1-21. In preferred embodiments, type II collagen binding proteins of the present invention with no detectable binding affinity for the Fc domain of immunoglobulins comprise one or more, preferably two or three, of the following amino acid residues in any of SEQ ID NOs: 2, 10, 11, 12, 14 and 15: aspartate (D) at the position corresponding to position 13 (13D) of any of SEQ ID NOs: 2, 10, 11, 12, 14 and 15; lysine (K) at the position corresponding to position 14 (14K) of any of SEQ ID NOs: 2, 10, 11, 12, 14 and 15; and/or arginine (R) at the position corresponding to position 31 (31R) of any of SEQ ID NOs: 2, 10, 11, 12, 14 and 15. Particularly preferred are type II collagen binding proteins with no detectable binding affinity for the Fc domain of immunoglobulins comprising one or more, preferably two or three, of the following amino acid residues in SEQ ID NO: 15: aspartate (D) at the position corresponding to position 13 (13D) of any of SEQ ID NO: 15; lysine (K) at the position corresponding to position 14 (14K) of SEQ ID NO: 15; and/or arginine (R) at the position corresponding to position 31 (31R) of SEQ ID NO: 15. Accordingly, the present invention provides a binding protein for type II collagen with no detectable binding affinity for the Fc domain of immunoglobulins, wherein the type II collagen binding protein comprises an amino acid sequence with at least 80% sequence identity to SEQ ID NO: 15, and comprises one or more, preferably two or three, of the following amino acid residues in SEQ ID NO: 15: aspartate (D) at the position corresponding to position 13 (13D) of any of SEQ ID NO: 15; lysine (K) at the position corresponding to position 14 (14K) of SEQ ID NO: 15; and/or arginine (R) at the position corresponding to position 31 (31R) of SEQ ID NO: 15. In various preferred embodiments, the type II collagen binding protein collagen with no detectable binding affinity for the Fc domain of immunoglobulins further comprises (i) a glutamine (Q) at the position corresponding to position 25 in SEQ ID NO: 15; (ii) a tyrosine (Y) at the position corresponding to position 29 in SEQ ID NO: 15; and (iii) a tryptophan (W) at the position corresponding to position 33 in SEQ ID NO: and 15; and has a binding affinity (K_{D}) of less than 10 μM for type II collagen, as described elsewhere herein.

In various embodiments of the present invention, the binding proteins for type II collagen with no detectable binding affinity for the Fc domain of immunoglobulin (or lacking binding affinity for the Fc domain of immunoglobulin) has no detectable binding affinity for the Fc domain of immunoglobulin (or lacks binding affinity for the Fc domain of immunoglobulin) as determined by methods for determining binding affinities described elsewhere herein. In preferred embodiments, the method for determining binding affinity to the Fc domain of immunoglobulin is any of ELISA, SPR, BLI, KinExA assay, flow cytometry, fluorescence spectroscopy techniques, ITC, analytical ultracentrifugation, RIA or IRMA, and ECL, more preferably any of ELISA, SPR, BLI, KinExA assay, and flow cytometry, even more preferably any of ELISA, SPR, and KinExA assay, still more preferably ELISA or SPR, and most preferred SPR.

The terms “Fc region” and “Fc domain” may be used interchangeably herein. The Fc region is the tail region of an immunoglobulin, in particular of an antibody that interacts for example with cell surface receptors called Fc receptors. Thus, the Fc region or Fc domain means the Fc region or Fc domain of an immunoglobulin, in particular an antibody. In various embodiments, the Fc region is from a mammalian IgG (antibody), including human IgG, mouse IgG, rat IgG, goat IgG, bovine IgG, guinea pig IgG, and rabbit IgG. The Fc region may also be from human IgM or human IgA. In various embodiments, the Fc region is from a human IgG (antibody), such as from a human IgG1 (antibody), human IgG2 (antibody), or human IgG4 (antibody), even more preferably from a human IgG1 (antibody).

Avimer™ domains are artificial proteins of 30-35 amino acids having a rigid structure stabilized in particular by disulfide bonds. In various embodiments of the present invention, a type II collagen binding protein as disclosed herein may or may not have an amino acid sequence that gives rise to disulfide bonds in the type II collagen binding proteins disclosed herein. Accordingly, in various embodiments of the present invention, a type II collagen binding protein as disclosed herein may or may not have an amino acid sequence comprising cysteine (Cys) residues that would give rise to disulfide bonds in the type II collagen binding proteins disclosed herein. More specifically, in various embodiments of the present invention, a type II collagen binding protein as disclosed herein may or may not have an amino acid sequence comprising two or more cysteine (Cys) residues that would give rise to one or more disulfide bonds in the type II collagen binding proteins disclosed herein. In various embodiments, a type II collagen binding protein as disclosed herein may or may not have an amino acid sequence not comprising any cysteine (Cys) residue(s).

Multimers. In preferred embodiments, the type II collagen binding protein is a multimer comprising of a plurality of the type II collagen binding protein as defined herein. A multimer may comprise two, three, four, or more type II collagen binding proteins. In one embodiment, the type II collagen binding protein comprises 2, 3, 4, or more type II collagen binding proteins linked to each other, i.e. the type II collagen-binding protein can be a dimer, trimer, or tetramer, etc. In some embodiments, the multimer is a dimer of the type II collagen binding protein as defined above.

In various embodiments, the type II collagen binding protein is a homo-dimer. Homo-dimeric type II collagen binding proteins are proteins wherein two type II collagen binding proteins with identical amino acid sequences are linked to each other. In some embodiments, the homo-multimeric type II collagen binding protein may comprise at least two modules of SEQ ID NO: 11 and has at least 85% sequence identity to the amino sequence of SEQ ID NO: 37 (211519).

In some embodiments, the homo-multimeric type II collagen binding protein may comprise at least two modules of SEQ ID NO: 15 and has at least 85% sequence identity to the amino sequence of SEQ ID NO: 40 (212705). For example, a homo-dimeric type II collagen binding protein may comprise two monomers of SEQ ID NO: 15 linked to each other in head-to-tail orientation. In some embodiments, the homo-multimeric type II collagen binding protein may comprise at least two modules of SEQ ID NO: 2 and has at least 85% sequence identity to the amino sequence of SEQ ID NO: 38 (211999). For example, a homo-dimeric type II collagen binding protein may comprise two monomers of SEQ ID NO: 2 linked to each other in head-to-tail orientation. In some embodiments, the homo-multimeric type II collagen binding protein may comprise at least two modules of SEQ ID NO: 1. For example, a homo-dimeric type II collagen binding protein may comprise two monomers of SEQ ID NO: 1 (211190) linked to each other in head-to-tail orientation.

In various embodiments, the type II collagen binding protein is a homo-tetramer. Tetrameric type II collagen binding proteins are proteins wherein four type II collagen binding proteins with identical amino acid sequences are linked to each other (see, for example, but not limited to, SEQ ID NO: 41 and SEQ ID NO: 42).

In other embodiments the multimer is a hetero-dimer, e.g. the two amino acid sequences of the type II collagen specific proteins have different amino acid sequences.

In some embodiments, a hetero-dimeric type II collagen binding protein may comprise SEQ ID NO: 10 and has at least 85% sequence identity to the amino sequence of SEQ ID NO: 10 (for example, the N-terminal monomer of the heterodimer of SEQ ID NO: 36 (210987), SEQ ID NO: 32 (210992), or SEQ ID NO: 33 (211027)).

n some embodiments, the hetero-dimeric binding protein may comprise SEQ ID NO: 14 and has at least 85% sequence identity to the amino sequence of SEQ ID NO: 14 (for example, SEQ ID NO: 28 (210970), the N-terminal monomer of the heterodimer of SEQ ID NO: 29 (210971), or the C-terminal monomer of the heterodimer of SEQ ID NO: 30 (210972) or SEQ ID NO: 31(210973).

For example, the hetero-dimeric binding protein of SEQ ID NO: 27 (209130) comprises SEQ ID NO: 4 and SEQ ID NO: 11 linked to each other in head-to-tail orientation (N- to C-terminus). For example, the hetero-dimeric binding protein of SEQ ID NO: 35 (208585) comprises SEQ ID NO: 17 and SEQ ID NO: 12 linked to each other in head-to-tail orientation (N- to C-terminus). For example, the hetero-dimeric binding protein of SEQ ID NO: 36 (210987) comprises SEQ ID NO: 10 and SEQ ID NO: 6 linked to each other in head-to-tail orientation (N- to C-terminus). Examples for dimeric type II collagen binding proteins are shown in FIG. 1C.

In some embodiments, two or more type II collagen binding proteins are directly linked. In some embodiments, two or more type II collagen binding proteins are linked by a peptide linker. In various embodiments, two or more type II collagen binding proteins are linked via a peptide linker of up to 30 amino acids. In other embodiments, two or more type II collagen binding proteins are linked via a peptide linker of 4, 5, 6, 7, 8, 9, 10, 11, 12, 13, 14, 15, 16, 17, 18, 19, 20 amino acids. Examples for linkers are provided in SEQ ID NOs: 45-48.

Multimers of the binding protein are generated artificially, generally by recombinant DNA technology well-known to a skilled person.

Type II collagen binding protein is fused to further moieties. In various embodiments, the type II collagen binding protein is fused to or conjugated to a therapeutically active moiety. In other embodiments, the type II collagen binding protein is fused to or conjugated to more than one therapeutically active moieties. In some embodiments, such therapeutically active moiety may be selected from a monoclonal antibody or a fragment thereof, an extracellular domain of a receptor or fragments thereof, a non-immunoglobulin scaffold protein, a cytokine, a chemokine, a cytotoxic compound, an enzyme, or derivatives thereof, a radionuclide, or any combination of the above.

In various embodiments, the type II collagen binding protein is fused to or conjugated to a diagnostic moiety. In other embodiments, the type II collagen binding protein is fused to or conjugated to more than one diagnostic moiety. In some embodiments, such diagnostic moiety may be selected from a radionuclide, a fluorescent compound, a photosensitizer, a tag, an enzyme, or a non-immunoglobulin scaffold protein, or any combination of the above.

Various embodiments relate to the type II collagen binding protein fused to a therapeutic active protein or a diagnostic active protein.

Use in medicine. Various embodiments relate to a type II collagen binding protein as disclosed herein for use in medicine. Specific embodiments relate to said type II collagen binding protein for use in the diagnosis or treatment of eye diseases and joint diseases. In some embodiments, eye diseases may be neovascular eye diseases. Neovascular eye diseases may be selected from the group of but not limited to neovascular age-related macular degeneration, myopic choroidal neovascularization, idiopathic choroidal neovascularization, choroidal neovascularization, branch retinal vein occlusion, central retinal vein occlusion, diabetic retinopathy, retinopathy of prematurity, diabetic macular edema. In some embodiments, eye diseases may be inflammatory eye diseases. Inflammatory eye diseases may be selected from the group of but not limited to non-infectious posterior uveitis, non-infectious intermediate uveitis, non-infectious anterior uveitis, idiopathic uveitis, non-infectious panuveitis, uveitis associated with primary inflammatory systemic disease such as Behcet's, sarcoid, lupus, juvenile idiopathic arthritis, rheumatoid arthritis, Wegener's granulomatosis, scleritis, and orbital inflammatory disease. Examples for joint related diseases are rheumatoid arthritis, arthrosis, and other joint related diseases.

Compositions. Various embodiments relate to a composition comprising the type II collagen binding protein as disclosed herein.

Various embodiments relate to a pharmaceutical composition for the treatment of diseases comprising the type II collagen binding protein as disclosed herein, and a pharmaceutically acceptable carrier and/or diluent. The pharmaceutical composition optionally may contain further auxiliary agents and excipients known per se. These include for example but are not limited to stabilizing agents, surface-active agents, salts, buffers, coloring agents etc. The compositions can be in the form of a liquid preparation, a lyophilisate, a cream, a lotion for topical application, an aerosol, in the form of powders, granules, in the form of an emulsion or a liposomal preparation.

The pharmaceutical composition comprising the type II collagen binding protein as defined herein can be used for treatment of eye diseases and joint diseases, as described above.

Various embodiments relate to a diagnostic composition for the diagnosis of diseases comprising the type II collagen binding protein as defined herein and a diagnostically acceptable carrier and/or diluent. These include for example but are not limited to stabilizing agents, surface-active agents, salts, buffers, coloring agents etc. The compositions can be in the form of a liquid preparation, a lyophilisate, granules, in the form of an emulsion or a liposomal preparation.

The diagnostic composition comprising the type II collagen binding protein as described herein can be used for diagnosis of eye diseases and joint diseases, as described above.

The compositions contain a therapeutically or diagnostically effective dose of the type II collagen binding protein as defined herein. The amount of protein to be administered depends on the organism to be treated, the type of disease, the age and weight of the patient and further factors known per se. Depending on the galenic preparation these compositions can be administered parentally by injection or infusion, systemically, intra-vitreous, intra-articular or by other conventionally employed methods of application. The type of pharmaceutical preparation depends on the type of disease to be treated, the route of administration, the severity of the disease, the patient to be treated and other factors known to those skilled in the art of medicine. Various embodiments relate to a method for treating or diagnosing a subject with an eye disease, comprising administering to the eye of the subject a therapeutically effective amount of the type II collagen binding protein as disclosed herein. Preferred is a method for treating a subject with an eye disease selected from retinal neovascular diseases such as neovascular age-related macular degeneration (AMD), diabetic macular oedema and retinal vein occlusion.

Preparation of type II collagen binding proteins. Type II collagen binding proteins as described herein may be prepared by any of the many conventional and well-known techniques such as plain organic synthetic strategies, solid phase-assisted synthesis techniques, fragment ligation techniques or by commercially available automated synthesizers. On the other hand, they may also be prepared by conventional recombinant techniques alone or in combination with conventional synthetic techniques. Furthermore, they may also be prepared by cell-free in-vitro transcription/translation or in combination with conventional synthetic techniques.

Various embodiments relate to a polynucleotide encoding a type II collagen binding protein as disclosed herein. The invention further provides an expression vector comprising said polynucleotide, and a host cell comprising said isolated polynucleotide or the expression vector.

Various embodiments relate to a method for the production of a type II collagen binding protein as disclosed herein comprising culturing of a host cell under suitable conditions which allow expression of said type II collagen binding protein and optionally isolating said type II collagen binding protein.

For example, one or more polynucleotides which encode for the type II collagen binding protein may be expressed in a suitable host and the produced type II collagen binding protein can be isolated. Vectors comprising said polynucleotides are covered herein. A further embodiment relates to a vector comprising said nucleic acid molecule. A vector means any molecule or entity (e.g., nucleic acid, plasmid, bacteriophage or virus) that can be used to transfer protein coding information into a host cell. Furthermore, an isolated cell is disclosed comprising said nucleic acid molecule or said vector. Suitable host cells include prokaryotes or eukaryotes. Various mammalian or insect cell culture systems can also be employed to express recombinant proteins.

An embodiment also relates to a host cell or a non-human host carrying said vector. A host cell is a cell that has been transformed with a nucleic acid sequence and thereby expresses a gene of interest.

Suitable conditions for culturing prokaryotic or eukaryotic host cells are well known to the person skilled in the art. Cultivation of cells and protein expression for the purpose of protein production can be performed at any scale, starting from small volume shaker flasks to large fermenters, applying technologies well-known to any skilled in the art.

One embodiment is directed to a method for the preparation of a binding protein as detailed above, said method comprising the following steps: (a) preparing a nucleic acid encoding a type II collagen binding protein as defined herein; (b) introducing said nucleic acid into an expression vector; (c) introducing said expression vector into a host cell; (d) cultivating the host cell; (e) subjecting the host cell to culturing conditions under which a type II collagen binding protein is expressed, thereby producing a type II collagen binding protein as defined herein; (f) optionally isolating the type II collagen binding protein produced in step (e); and (g) optionally conjugating the type II collagen binding protein with further functional moieties as defined herein.

In general, isolation of purified type II collagen binding protein from the cultivation mixture can be performed applying conventional methods and technologies well known in the art, such as centrifugation, precipitation, flocculation, different embodiments of chromatography, filtration, dialysis, concentration and combinations thereof, and others. Chromatographic methods are well-known in the art and comprise without limitation ion exchange chromatography, gel filtration chromatography (size exclusion chromatography), hydrophobic interaction chromatography, or affinity chromatography.

For simplified purification, the type II collagen binding protein can be fused to other peptide sequences having an increased affinity to separation materials. Preferably, such fusions are selected that do not have a detrimental effect on the functionality of the type II collagen binding protein or can be separated after the purification due to the introduction of specific protease cleavage sites. Such methods are also known to those skilled in the art.

### EXAMPLES

The following Examples are provided for further illustration of the invention. The invention is particularly exemplified by type II collagen binding proteins as described above. The invention, however, is not limited thereto, and the following Examples merely show the practicability of the invention on the basis of the above description. For a complete disclosure of the invention reference is made also to the literature cited in the application which is incorporated completely into the application by reference.

### Example 1. Selection and Screening of Type II Collagen Alpha I Chain Binding Proteins

Libraries. Proprietary cDNA libraries were synthesized in house by randomized oligonucleotides generated by synthetic trinucleotide phosphoramidites (ELLA Biotech) to achieve a well-balanced amino acid distribution with simultaneously exclusion of cysteine and other amino acid residues at randomized positions. The corresponding cDNA library was amplified by PCR and ligated into a pCD33-OmpA phagemid. Aliquots of the ligation mixture were used for electroporation of E. coli SS320 (Lucigen) to produce and purify the phage library and to store cryo-stocks of it. Unless otherwise indicated, established recombinant genetic methods were used.

Selection by phage display. native libraries were enriched against the respective ON-target (Immunization Grade Human Type II Collagen, purchased from Chondrex Inc.) using phage display as selection system. In each round a pre-selection step was performed using empty Sigmablocker-blocked beads as OFF-target. The AIT-method was applied, which means that the target proteins were immobilized to magnetic Epoxy M-270 Dynabeads for each round. E. coli ER2738 (Lucigene) were used for infection with cryo phage libraries and for reamplification of phage pools after each round. Amplification and purification of the phages were carried out using standard methods known to a skilled person. Four selection rounds were performed with the automated KingFisher-System (Thermo Fisher) to isolate and capture the desired phage-target complexes. The target amount was reduced from round to round by lowering the volume of target-coupled beads. Bound phages were eluted by trypsin and reamplified. The success of the selection was analyzed by phage-pool-ELISA in medium binding microtiter plates (Greiner Bio-One) coated with Immunization Grade Human Type II Collagen (Chondrex Inc., 5 μg/ml), porcine collagen type II (in-house prepared from pig eyes, 5 μg/ml), BSA (5 μg/ml) or Sigmablocker. Bound phages were detected using α-M13 HRP conjugated antibody (GE Healthcare).

Cloning of target binding phage pools into an expression vector. Selection pools showing specific binding to human and/or porcine collagen type II in phage pool ELISA were amplified by PCR according to methods known in the art, cut with appropriate restriction nucleases and ligated into a derivative of the expression vector pET-28a (Merck, Germany) comprising a C-terminal strep-tag.

Results: Various phage display selection pools resulted in specific signals for the respective ON-target. Controls with BSA and Sigmablocker showed no binding for most of the pools. Selected pools were sequenced, subcloned and proceed to high throughput screening.

Primary screening: Primary screening of single variants vs. direct immobilized Immunization Grade Human Type II Collagen (Chondrex Inc.) [c=1 μg/ml] was performed. Bound variants were detected using Strep-Tactin® HRP Conjugate. Hits were selected as follows: signal of sample larger than signal of negative control.

Secondary screening. Secondary screening versus Immunization Grade Human Type II Collagen (Chondrex Inc.; on-target) [c=1 μg/ml] and BSA (off-target) [c=1 μg/ml] was performed. Hits were identified as follows: signal of variants vs. on-target is tenfold larger than signal vs. off-target.

PhyNexus/SPR. Hits were produced and purified in p-scale (PhyNexus) and analyzed by SPR measurement. Immunization Grade Human Type II Collagen (Chondrex Inc.) was immobilized on a CM5 chip (GE Helthcare) via amine coupling. Upon binding, analyte (possible hit) was accumulated on the surface increasing the refractive index. The change of the refractive index was measured in real time and plotted as response unit versus time. Hit variants were selected for sequence analysis and lab scale production.

### Example 2. Expression of Type 2 Collagen Binding Proteins

Variants with affinity tag were expressed in Escherichia coli BL21(DE3) using a low copy plasmid system under regulation of a T7 promoter. Proteins were produced in soluble form after induction by lactose included in the medium (autoinduction medium). BL21 (DE3) competent cells were transformed with the expression plasmid, spread onto selective agar plates (kanamycin) and incubated overnight at 37° C. Precultures were inoculated from single colony in 3 ml 2×YT medium supplemented with 50 μg/ml kanamycin and cultured for 6 hours at 37° C. at 200 rpm in a conventional orbital shaker in culture tubes. Main cultures were inoculated with 0.3/OD of precultures in 350 ml ZYM-5052 (0.5% glycerol, 0.2% lactose, 0.05% glucose, 0.5% yeast extract, 1.0% casamino acids, 25 mM Na₂HPO₄, 25 mM KH₂PO₄, 5 mM Na₂SO₄, 2 mM MgSO₄ and trace elements) for variants with affinity tag or 350 ml H15 medium (0.89% glycerol, 0.76% lactose, 2.2% glucose, 5% yeast extract, 250 mM MOPS, 200 mM Tris, 10 mM MgCl2 and trace elements) for variants without affinity tag that was supplemented with 50 μg/ml kanamycin in 1 L Erlenmeyer flasks. Cultures were transferred to an orbital shaker and incubated at 30° C. and 200 rpm. Recombinant protein expression was induced by metabolizing glucose and subsequently allowing lactose to enter the cells. Cells were grown overnight for approx. 17 hours to reach a final OD600 of about 2-4. Before the harvest, the OD600 was measured, samples adjusted to 0.6/OD600 were withdrawn, pelleted and frozen at −20° C. To collect biomass cells were centrifuged at 12000×g for 15 min at 22° C. Pellets were weighed (wet weight). Cells were stored at −20° C. before processing.

### Example 3. Purification of Type 2 Collagen Binding Proteins

Proteins with affinity tag were purified by affinity chromatography and size exclusion. After affinity chromatography purification using Strep-Tactin Superflow high capacity cartridges (IBA Lifesciences) a size exclusion chromatography (SEC) was performed using an Äkta system and a Superdex™ 200 HiLoad 16/600 column (Cytiva). Fused variants with Aflibercept were purified via MabSelect SuRe (Cytiva) affinity chromatography followed by a size exclusion chromatography (SEC) using an Äkta system and a Superdex™ 200 HiLoad 16/600 column (Cytiva). All chromatography processing steps were carried out on AKTA systems using matrices from Cytiva. Homogenous species of all purified ligands were obtained; no aggregation was detected. Following SOS-PAGE analysis positive fractions were pooled and their protein concentrations were measured. Further analysis included SOS-PAGE, SE-HPLC and RP-HPLC. Protein concentrations were determined by absorbance measurement at 280 nm using the molar absorbent coefficient. Reversed phase chromatography (RP-HPLC) was performed using a Dionex HPLC system and a PLRP-S (5 μm, 300 Å) column (Agilent). Analytic size exclusion chromatography (SE-HPLC) was performed using a Dionex HPLC system and a Superdex200 increase 5/150 GL (Cytiva). Binding of Collagen II was evaluated by SPR measurements using a Biacore 3000 system. Purified Collagen II was obtained from Chondrex.inc and VEGF-165 was obtained from Millipore. Targets were immobilized on a CM5-Sensor via NHS/EDC chemistry (immobilization level ˜1000 RU). Binding molecules were injected at different concentration and affinity was calculated by langmuir 1:1 binding model. TABLE 2 shows the results of the expression and purification of selected type 2 collagen binding proteins (or multimers comprising such proteins).

TABLE 2
Expression and purification of Type II collagen binding proteins

| | | | Solubility | Yield Per | Yield Per | |
|---|---|---|---|---|---|---|
| SEQ ID | | SE-HPLC | Soluble | Volume | Weight | rp-HPLC |
| NO: | CID | Monomer | expression | Yield mg/L | Yield mg/g | Main peak |
| 1 | 211190 | 100% | 95% | 25.51 | 1.86 | 97% |
| 2 | 211261 | 100% | 95% | 7.86 | 0.55 | 48% |
| 37 | 211519 | 100% | 95% | 9.40 | 0.43 | 59% |
| 27 | 209130 | 84% | 90% | 14.0 | n.d. | n.d. |
| 36 | 210987 | 100% | 80% | 39.77 | 2.78 | 78% |
| 28 | 210970 | 100% | 90% | 27.43 | 1.55 | 78% |
| 29 | 210971 | 100% | 60% | 2.55 | 0.13 | 43% |
| 30 | 210972 | 100% | 70% | 3.98 | 0.23 | 50% |
| 31 | 210973 | 100% | 90% | 5.38 | 0.28 | 59% |
| 32 | 210992 | 100% | 95% | 39.19 | 2.80 | 62% |
| 33 | 211027 | 100% | 85% | 44.65 | 3.26 | 68% |
| 34 | 211138 | 100% | 95% | 52.18 | 3.38 | 55% |
| 38 | 211999 | 100% | 100% | 3.00 | 0.19 | 53% |
| 39 | 212000 | 100% | 100% | 5.08 | 0.33 | 61% |
| 40 | 212705 | 100% | 100% | 16.69 | 0.89 | 74% |
| 41 | 212001 | 100% | 100% | 7.66 | 0.51 | 100% |
| | (tetramer) | | | | | |
| 42 | 212704 | 100% | 100% | 46.03 | 2.34 | 100% |
| | (tetramer) | | | | | |

### Example 4. Binding Analysis of Proteins by SPR

The purified proteins were immobilized on a CM-5 sensor chip (GE Healthcare) using NHS/EDC after PDEA activation resulting in 110-140 RU with a Biacore 3000 system (GE Healthcare). The chip was equilibrated with SPR running buffer (PBS 0.05% Tween pH 7.3). Upon binding, target analyte was accumulated on the surface increasing the refractive index. This change in the refractive index was measured in real time and plotted as response or resonance units versus time. The analytes were applied to the chip in serial dilutions with a flow rate of 30 μl/min. The association was performed for 120 seconds and the dissociation for 120 seconds. After each run, the chip surface was regenerated with 30 μl regeneration buffer (10 mM glycine pH 2.0) and equilibrated with running buffer.

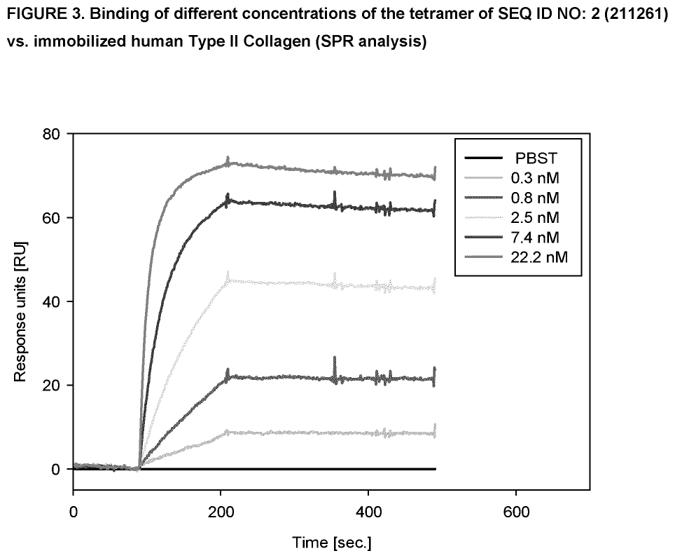

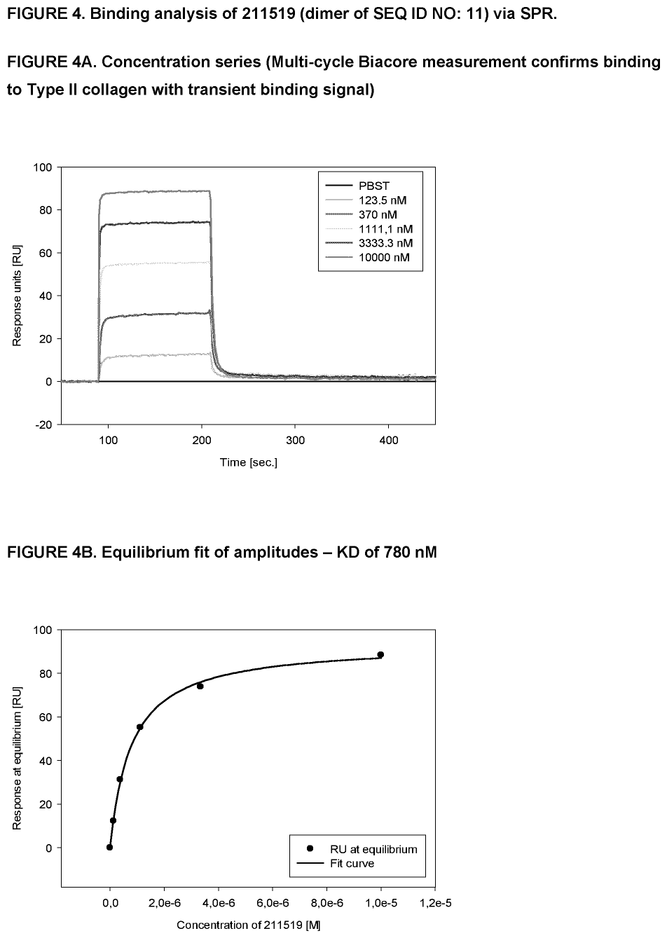

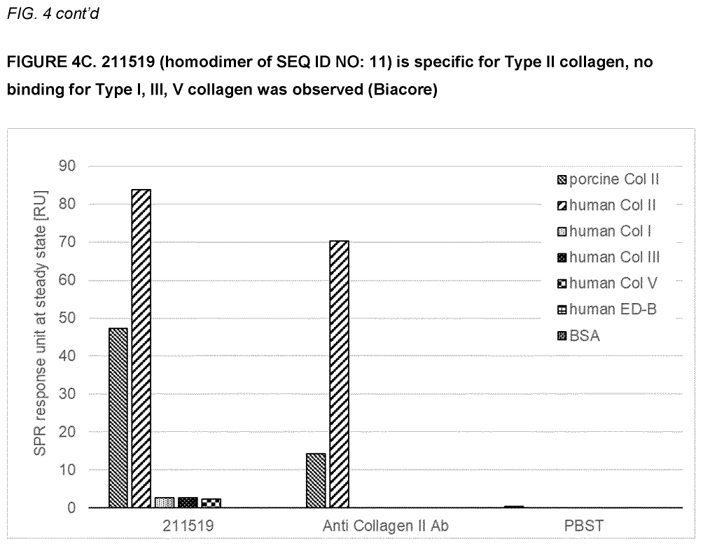

Binding studies were carried out by the use of the BIAcore 3000 (GE Healthcare); data evaluation was operated via the BIAevaluation 3.0 software, provided by the manufacturer, using the Langmuir 1:1 model (RI=0). Evaluated dissociation constants (K_{D}) were standardized against the immobilized protein and indicated. Shown is the change in refractive index measured in real time and plotted as response or resonance unit [RU] versus time [sec]. Results are shown in TABLE 3 and FIG. 3 and FIG. 4. 211190 (SEQ ID NO: 1) showed additional binding (additional to Type II collagen) to Type I collagen and Type III collagen, but not to Type V collagen (and not to controls, BSA or extradomain B of fibronectin).

TABLE 3
Affinity (SPR) of type 2 collagen binding
proteins (dimers or tetramers)

| SEQ ID | | Affinity [SPR] vs | |
|---|---|---|---|
| NO: | CID | hCol2 | |
| 37 | 211519 | 784 | nM |
| 36 | 210987 | 169 | nM |
| 27 | 209130 | n.d. | |
| 38 | 211999 | 127 | nM |
| 39 | 212000 | 180 | nM |
| 41 | 212001 | <0.5 | nM |
| 42 | 212704 | 0.66 | nM |

### Example 5. K_{D}-Determination of Binding Proteins (ELISA)

High binding plates (Greiner, 781061) were immobilized with 2.5 μg/ml recombinant human collagen type II (h Col II, Chondrex, CHX-20051), rabbit collagen type II (rbCol II, prepared from rabbit eyes), porcine collagen type II (p Col II, prepared from porcine eyes), human collagen type I, (h Col I, Sigma, C5483-1MG), human collagen type V (h Col V, Abcam, ab7537), human collagen type III (h Col Ill, Abcam, ab7535) and 67B89 (domain of fibronectin, produced in E. coli) respectively over night at 4° C. ELISA-plates were washed 3 times with PBST (PBS+ 0.1% Tween) and blocked with 3% BSA/0.5% Tween/PBS 2 h at RT. After washing with PBST, dilution series of 1.1 μM to 7×10⁻⁴ nM of dimers (CID 211999, CID 212000), dilution series of 100 nM to 7×10⁻⁶ nM of tetramers (CID 212001 and CID 212704) and dilution series of 10 μM to 2 nM of dimer (CID 212705) were incubated on ELISA-plates 1 h at rt. Plates were washed with PBST and incubated with biotinylated anti-StrepTag-ab (Antikörper-online, ABIN1573895) with a dilution of 1:10.000 for 1 h at rt. The binding was visualized with a-rabbit-IgG-HRP (Sigma, A9169) with a dilution of 1:10.000 and as substrate TMB Plus von KEM EN TEC Diagnostic, Kat-Nr. 4395.

Results. ELISA analysis clearly confirms the specific binding to type II collagen. Homodimers of SEQ ID NO: 2 (CID 211999 and CID 212000) and a homodimer of SEQ ID NO: 15 (CID 212705) show a high binding affinity (K_{D} of 1-2-digit nM). The tetramer of SEQ ID NO: 15 (CID 212704) and the tetramer of SEQ ID NO: 2 (CID 212001) show a high binding affinity, e.g. a 3 digit-picomolar binding affinity to type II collagen of different species. No unspecific binding to type I collagen, type III collagen, type V collagen or to a domain of fibronectin (67B89) was detected (FIG. 2).

Further Type II collagen binding proteins were analysed by ELISA for binding. For example, 209130 (SEQ ID NO: 27) showed specific binding of 0.22 μM vs human type II collagen and 0.11 μM vs porcine type II collagen. 211138 (SEQ ID NO: 34) showed specific binding of 0.45 μM vs human type II collagen and 0.81 μM vs porcine type II collagen. 210987 (SEQ ID NO: 36) showed specific binding of 0.46 μM vs human type II collagen. 211519 (SEQ ID NO: 37) showed specific binding of 0.7 μM vs human type II collagen and vs. porcine type II collagen. 211190 (SEQ ID NO: 1) showed specific binding vs human type II collagen, and additionally to human type I collagen, human type III collagen, but not to human type V collagen.

### Example 6: Analysis of Specific Binding of Proteins by IHC

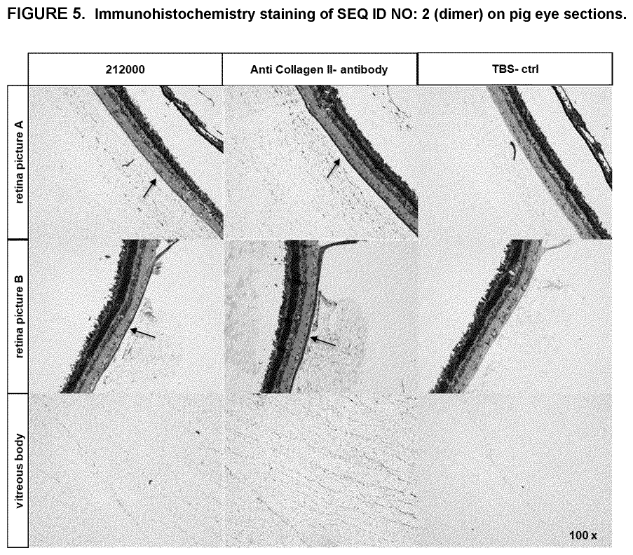

Sections of formalin-fixed paraffin-embedded pig eyes were deparaffinated with xylol and rehydrated with a descending concentration series of ethanol. For antigen retrieval slices were incubated in TE-buffer, pH 9.0 for 5 min. at 110° C. After blocking of endogenous peroxidases with peroxidase blocking solution (Novolink Polymer™ Detection System, Leica, RE7140-K) slices were incubated with 5 μM or 500 nM of CID 211999 (dimer), CID 212000 (dimer), CID 212001 (tetramer) for 45 min at rt. Subsequently binding of proteins was detected with rabbit-anti-StrepTag-antibody (Genscript, A00026) at a dilution of 1:500 and Novolink Polymer™ Detection System. Anti-Collagen Type II antibody (Abcam, #ab34712) at a dilution of 1:10 served as positive control. Tissue slices were incubated with antibodies for 30 min. Washing steps were performed with TBS pH 7.6. For visualization, AEC-staining (Dako, K3461) was used and after washing with water, nuclei were stained with Mayers Hamalum solution (Merck, 1.09249.0500). Results see FIG. 5. Similar results as those shown in FIG. 5 were obtained with SEQ ID NO: 38 (dimerSEQ ID NO: 2, CID 211999) and SEQ ID NO: 41 (tetramer of SEQ ID NO: 2, CID 212001).

1. A type II collagen binding protein comprising an amino acid sequence with at least 80% sequence identity to any one of SEQ ID NOs: 2, 10, 11, 12, 14, and 15, wherein the type II collagen protein has
(i) a glutamine (Q) at the position corresponding to position 25 in any of SEQ ID NOs: 2, 10, 11, 12, 14 and 15;
(ii) a tyrosine (Y) at the position corresponding to position 29 in any of SEQ ID NOs: 2, 10, 11, 12, 14 and 15; and
(iii) a tryptophan (W) at the position corresponding to position 33 in any of SEQ ID NOs: 2, 10, 11, 12, 14 and 15;
and wherein the type II collagen binding protein has a binding affinity (K_{D}) of less than 10 μM for type II collagen.

2. The binding protein for type II collagen according to claim 1, wherein 2, 3, 4, 5, or 6 binding proteins for type II collagen are linked to each other.

3. The binding protein for type II collagen according to claim 2, wherein the binding protein is a homo-multimer or a hetero-multimer.

4. The binding protein for type II collagen according to claim 2, wherein the binding protein comprises a first domain comprising an amino acid sequence with at least 80% sequence identity to any one of SEQ 2, 10-12, 14, and 15, a second domain with an amino acid sequence with at least 80% sequence identity to any one of SEQ ID NO: 2-4, 6-8, 10-12, 14, 15, 17, 18, 20, and 21, or both, optionally wherein the binding protein for type II collagen comprises an amino acid sequence with at least 80% identity to any one of SEQ ID NOs: 27-40.

5. A dimeric or multimeric protein comprising the type II collagen binding protein according to claim 4.

6. The type II collagen binding protein according to claim 1, wherein the type II collagen binding protein is fused to at least one therapeutically active moiety, optionally wherein the at least one therapeutically active moiety is selected from the group consisting of a monoclonal antibody or a fragment thereof, a binding protein, a receptor or receptor domain, a receptor binding ligand or antagonist, an extracellular domain of a receptor or fragments thereof, a non-immunoglobulin binding protein, a cytokine, a chemokine, a cytotoxic compound, an enzyme, and a radionuclide, or any combination thereof.

7. The type II collagen binding protein according to claim 1, wherein the type II collagen binding protein is fused to or conjugated to at least one diagnostic moiety, optionally wherein the diagnostic moiety is selected from the group consisting of a radionuclide, a fluorescent compound, a photosensitizer, a tag, an enzyme, and a non-immunoglobulin binding protein, or any combination thereof.

8-9. (canceled)

10. A pharmaceutical composition comprising the type II collagen binding protein of claim 1 and a therapeutically or diagnostically acceptable carrier and/or diluent.

11. A method for producing the type II collagen binding protein of claim 1, comprising culturing a host cell comprising a nucleic acid sequence that encodes the type II collagen binding protein under suitable conditions in order to obtain said type II collagen binding protein, and optionally isolating said type II collagen binding protein from the host cell or from media in which the host cell is growing.

12. The type II collagen binding protein according to claim 4, wherein the type II collagen binding protein is fused to or conjugated to at least one diagnostic moiety, optionally wherein the diagnostic moiety is selected from the group consisting of a radionuclide, a fluorescent compound, a photosensitizer, a tag, an enzyme, and a non-immunoglobulin binding protein, or any combination thereof.

13. The dimeric or multimeric protein according to claim 5, wherein the dimeric or multimeric protein is fused to or conjugated to at least one therapeutically active moiety, optionally wherein the at least one therapeutically active moiety is selected from the group consisting of a monoclonal antibody or a fragment thereof, a binding protein, a receptor or receptor domain, a receptor binding ligand or antagonist, an extracellular domain of a receptor or fragments thereof, a non-immunoglobulin binding protein, a cytokine, a chemokine, a cytotoxic compound, and an enzyme, and/or is labeled with a radionuclide, or any combination thereof.

14. The dimeric or multimeric protein according to claim 5, wherein the dimeric or multimeric protein is fused to or conjugated to at least one diagnostic moiety, optionally wherein the diagnostic moiety is selected from the group consisting of a radionuclide, a fluorescent compound, a photosensitizer, a tag, an enzyme, and a non-immunoglobulin binding protein, or any combination thereof.

15. A pharmaceutical composition comprising the dimeric or multimeric protein according to claim 5 and a therapeutically or diagnostically acceptable carrier and/or diluent.

16. A method for producing the dimeric or multimeric protein of claim 5, comprising culturing a host cell comprising a nucleic acid sequence that encodes the dimeric or multimeric protein under suitable conditions in order to obtain said dimeric or multimeric protein, and optionally isolating said dimeric or multimeric protein from the host cell or from media in which the host cell is growing.
